(19) United States
(12) Patent Application Publication
(10) Pub. No.: US 2024/0033389 A1
(43) Pub. Date: Feb. 1, 2024

(54) SYSTEM AND METHOD FOR DETECTING PERACTIC ACID AND HYDROGEN PEROXIDE VAPOR

(71) Applicants: John Matta, Shoreview, MN (US); Kristopher Murphy, New Brighton, MN (US); Huyen Bui, Brooklyn Park, MN (US); Tuan Nguyen, Chaska, MN (US); Sherly Bellevue Faye, (US); Manson Schwartz, Elk River, MN (US); Ted Bahns, White Bear Lake, MN (US); Lisa Bourdon, Waconia, MN (US)

(72) Inventors: John Matta, Shoreview, MN (US); Kristopher Murphy, New Brighton, MN (US); Huyen Bui, Brooklyn Park, MN (US); Tuan Nguyen, Chaska, MN (US); Sherly Bellevue Faye, (US); Manson Schwartz, Elk River, MN (US); Ted Bahns, White Bear Lake, MN (US); Lisa Bourdon, Waconia, MN (US)

(21) Appl. No.: 18/159,796

(22) Filed: Jan. 26, 2023

Related U.S. Application Data
(62) Division of application No. 16/771,265, filed on Jun. 10, 2020, now abandoned.
(60) Provisional application No. 62/608,798, filed on Dec. 21, 2017.

(51) Int. Cl. A61L 2/28; A61B 90/70; A61L 2/20; G01N 21/3504; G01N 21/359

(52) U.S. Cl. CPC A61L 2/28; A61B 90/70; A61L 2/208; G01N 21/3504; G01N 21/359; A61B 2090/701; A61B 2090/702; A61L 2202/14; A61L 2202/24

(57) ABSTRACT

The present invention relates to the detection vapor peracetic acid and hydrogen peroxide. It finds particular application in the sensing of vapor peracetic acid and hydrogen peroxide concentrations. The system includes (a) a source of peracetic acid vapor, hydrogen peroxide vapor, water vapor and acetic acid vapor, (b) a light source which is configured to supply light with at least a component in the mid-infrared range, and (c) a detector which is configured to individually detect mid-infrared range light in (i) a first mid-infrared spectrum absorbed by the peracetic acid vapor and not absorbed by the hydrogen peroxide vapor, the acetic acid vapor or the water vapor, and (ii) a second mid-infrared spectrum absorbed by the peracetic acid vapor and the hydrogen peroxide vapor.

### PRIORITY CLAIM

This application is a divisional of U.S. patent application Ser. No. 16/771,265, filed Jun. 10, 2020, which is a 371 of international PCT Application with serial number PCT/US18/66851 filed Dec. 20, 2018, which claims priority to and the benefit of U.S. Provisional application with serial number 62/608,798, filed on Dec. 21, 2017, entitled SYSTEM AND METHOD FOR DETECTING PERACTIC ACID AND HYDROGEN PEROXIDE VAPOR, the contents of which are herein incorporated in their entirety for all purposes.

### TECHNICAL FIELD

The present invention relates to the detection vapor peracetic acid (PAA) and hydrogen peroxide. It finds particular application in the sensing of vapor peracetic acid and hydrogen peroxide concentrations.

### BACKGROUND OF INVENTION

Advanced medical instruments formed of rubber and plastic components with adhesives are delicate and often unsuited to the high temperatures and pressures associated with a conventional steam autoclave. Steam autoclaves often operate under pressure cycling programs to increase the rate of steam penetration into the medical devices or associated packages of medical devices undergoing sterilization. Steam sterilization using gravity, high pressure, or pre-vacuum, creates an environment where rapid changes in temperature or pressure can take place. Complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

Endoscopes can present certain problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes.

Decontamination systems and methods that utilize peracetic acid and/or hydrogen peroxide chemistry are known. For example, PCT Patent Application No. PCT/US17/59670 and U.S. Patent Application 2016/0346416 both of which are incorporated by reference in their entirety, disclose decontamination or sterilization systems that utilize peracetic acid and/or hydrogen peroxide.

While current systems set cycle parameters to avoid oversaturation of the vapor, the saturation of the process has not typically been monitored or controlled.

### SUMMARY OF INVENTION

There is a need for a system and method for detecting the presence and concentration of peracetic acid vapor and hydrogen peroxide vapor, preferably during a sterilization or decontamination cycle, in order to verify the presence and/or efficacy of the cycle.

In one aspect, the present invention is directed to a peracetic acid vapor and hydrogen peroxide vapor detection system. The system includes (a) a source of peracetic acid vapor, hydrogen peroxide vapor, water vapor and acetic acid vapor, (b) a light source which is configured to supply light with at least a component in the mid-infrared range, and (c) a detector which is configured to individually detect mid-infrared range light in (i) a first mid-infrared spectrum absorbed by the peracetic acid vapor and not absorbed by the hydrogen peroxide vapor, the acetic acid vapor or the water vapor, and (ii) a second mid-infrared spectrum absorbed by the peracetic acid vapor and the hydrogen peroxide vapor.

In another aspect, the present invention is directed to a peracetic acid and hydrogen peroxide treatment system. The system includes (a) a treatment chamber, (b) a vaporizer configured for generating a mixture of peracetic acid vapor, hydrogen peroxide vapor, water vapor and acetic acid vapor and supplying the vapor mixture to the treatment chamber, (c) a light source which is configured to supply light to the treatment chamber with at least a component in the mid-infrared range, (d) a detector which individually detects mid-infrared range light in a first spectrum absorbed by peracetic acid vapor and not any of the hydrogen peroxide vapor, water vapor and acetic acid vapor, and a second spectrum absorbed by the peracetic acid vapor and the hydrogen peroxide vapor, and (e) a processor configured to determine the concentration of the peracetic acid vapor in the treatment chamber.

In another aspect, the present invention is directed to a disinfection or sterilization system. The system includes (a) a treatment chamber, (b) a vaporizer configured to vaporize an aqueous solution comprising peracetic acid, hydrogen peroxide, acetic acid and water to form a mixture of peracetic acid vapor, a hydrogen peroxide vapor, an acetic acid vapor and a water vapor and for supplying the mixture of vapors to the treatment chamber, (c) a light source which is configured to project a beam of light in a mid-infrared range through the mixture of vapors, (d) a mid-infrared light detector which is configured to detect a first spectrum absorbed by the peracetic acid vapor and not any of the hydrogen peroxide vapor, the acetic acid vapor and the water vapor, and a second spectrum absorbed by the peracetic acid vapor and the hydrogen peroxide vapor, (e) a first processor which is configured to convert the detected first and second spectrum light into one of (i) absorbance values indicative of mid infrared light absorbed by the peracetic acid and hydrogen peroxide vapors and (ii) transmittance values indicative of mid-infrared light transmitted through the peracetic acid and hydrogen peroxide vapors, and (f) a second processor which is configured to convert the determined absorbance or transmittance values into a concentration of the peracetic acid vapor and a concentration of the hydrogen peroxide vapor.

In another aspect, the present invention is directed to a method for detecting the presence of peracetic acid and hydrogen peroxide in a vapor mixture. The method includes the steps of a) providing a vaporized mixture comprising peracetic acid, hydrogen peroxide, acetic acid, and water into a chamber, b) projecting light in a mid-infrared range through a portion of the vaporized mixture that has passed through at least a portion of the chamber, (c) detecting mid-infrared light in a first spectrum absorbed by the peracetic acid vapor and not any of the hydrogen peroxide vapor, the acetic acid vapor and the water vapor, and a second narrow spectrum absorbed by a peracetic acid vapor and hydrogen peroxide vapor, and (d) detecting mid-infrared light in a second spectrum absorbed by the peracetic acid vapor and the hydrogen peroxide vapor.

In another aspect, the present invention is directed to a method for detecting the presence of peracetic acid and hydrogen peroxide in a vapor mixture. The method includes the steps of (a) providing a vaporized mixture comprising peracetic acid, hydrogen peroxide, acetic acid, and water into a chamber, (b) projecting light in a mid-infrared range through a portion of the vapor mixture that has passed through a portion of the chamber, (c) detecting mid-infrared light in a first spectrum absorbed by the peracetic acid vapor and not any of the hydrogen peroxide vapor, the acetic acid vapor and the water vapor, and a second narrow spectrum absorbed by a peracetic acid vapor and hydrogen peroxide vapor, (d) projecting light in a near-infrared range through the monitored region of the chamber, and (e) detecting near-infrared light in a spectrum absorbed by the peracetic acid vapor, the hydrogen peroxide vapor and the acetic acid vapor.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

The invention may take form in various components and arrangements of components, and in various steps and arrangements of steps. The drawings are only for purposes of illustrating preferred embodiments and are not be construed as limiting the invention.

FIG. 1 is an exemplary mid-infrared spectrum of the peracetic acid (PAA)/hydrogen peroxide(H₂O₂)/acetic acid (AA)/water system.

FIG. 2 is a graph showing the relationship between PAA vapor concentration and peak intensity in the region from 840 cm⁻¹ to 880 cm⁻¹ from FIG. 1.

FIG. 3 is a graph showing the relationship between PAA vapor concentration and peak intensity in the region from 1230 cm⁻¹ to 1250 cm⁻¹ from FIG. 1.

FIG. 4 is an exemplary near-infrared spectrum of the peracetic acid/hydrogen peroxide/acetic acid/water system.

FIG. 5 shows a graph of pressure versus time within an exemplary decontamination or sterilization chamber in an example embodiment of a decontamination or sterilization cycle.

FIG. 6 is the mid-infrared spectrum of the peracetic acid/hydrogen peroxide/acetic acid/water system of Example 1.

FIG. 7 is a schematic illustrating the setup of sampling collection for Example 3

FIG. 8 shows a graph of the calculated PAA vapor (mg/L) and PAA absorption calibration curve with the injection volumes at operating pressure of 100 torr for the data in Table 2 of Example 3.

FIG. 9 shows a graph of the calculated PAA vapor (mg/L) and PAA absorption calibration curve with the injection volumes at operating pressure of 100 torr for the data in Table 3 of Example 3.

FIG. 10 shows a graph of the calculated PAA vapor (mg/L) and PAA absorption calibration curve with the injection volumes at operating pressure of 75 torr for the data in Table 4 of Example 3.

FIG. 11 shows a graph of the calculated PAA vapor (mg/L) and PAA absorption calibration curve with the injection volumes for the accumulated data Tables 2, 3 and 4 of Example 3.

### DETAILED DESCRIPTION OF THE INVENTION

Devices, such as medical devices, can be decontaminated or sterilized at relatively low temperatures using vaporized mixture of peracetic acid, hydrogen peroxide, acetic acid and water. In such systems, the chemistry may be provided as a vapor into a decontamination chamber containing the device to be decontaminated. The surfaces of the device will be decontaminated when contacted with the chemistry. Lumen devices may be particularly challenging to decontaminate as there must be flow of the decontaminating substance through the lumen. The instant disclosure describes a system for detecting the presence and/or concentration of peracetic acid vapor, hydrogen peroxide vapor and optionally, acetic acid vapor during the decontamination or sterilization process. A method of using is also described.

The vapor detection systems and methods of the present invention can be used alone or in combination with sterilization or decontamination systems, such as those disclosed in PCT Patent Application No. PCT/US17/59670 and U.S. Patent Application 2016/0346416 both of which are incorporated by reference in their entirety.

The present invention is directed to a system and method which includes detecting the absorbance of the vapor mixture (peracetic acid vapor, hydrogen peroxide vapor, acetic acid vapor and water vapor), for example, by passing the mixture through a gas cell, in the mid-infrared (MIR) range (which is defined as 4000 cm⁻¹ to 400 cm⁻¹), and also optionally in the near infrared (NIR) range (700 nm to 2500 nm). The system or method, or various components of the system or method, can be located or carried out inside of a decontamination or sterilization chamber, or outside of the chamber.

In one embodiment, the system is a detection system. In another embodiment, the system is a treatment system. The treatment system can be disinfection or sterilization and can be used for medical devices, such as endoscopes.

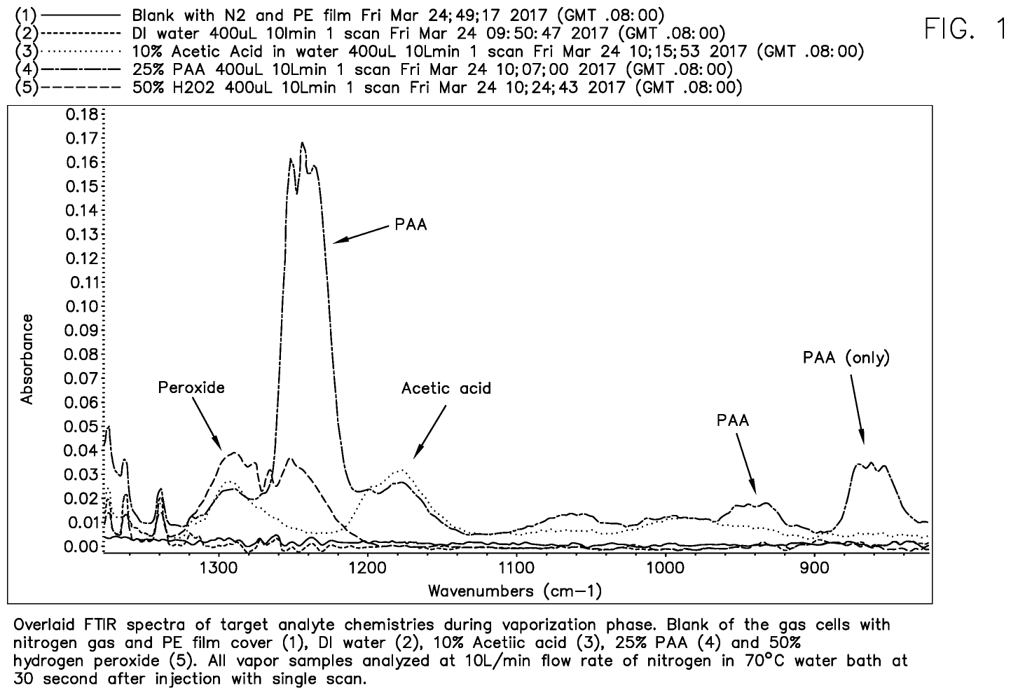

An exemplary mid-infrared spectrum of the peracetic acid/hydrogen peroxide/acetic acid/water system is shown in FIG. 1. FIG. 1 shows an overlaid FTIR spectra of target analyte chemistries during a vaporization phase. The blank reading indicates the gas cell with nitrogen gas and a polyethylene film cover. The DI water reading indicates the gas cell filled with DI water. The PAA reading is for a 25% PAA solution. The Acetic acid reading is for a 10% acetic acid solution. The Peroxide reading is for a 50% hydrogen peroxide solution. All vapor samples were analyzed at a flow rate of 10 mL/minute of nitrogen in a 70° C. water bath at 30 seconds after injection with a single scan.

It is believed that the absorbance of the peracetic acid band which shows a set of peaks between 1200 cm⁻¹ and 1140 cm⁻¹ in FIG. 1 is due to a residual contamination of the PAA with some acetic acid.

As shown in FIG. 1, the PAA absorbance is present as a triplet of peaks between 830 cm⁻¹ and 880 cm⁻¹. In this region, with this 4-component system (hydrogen peroxide, acetic acid, peracetic acid, water), the region between 830 cm⁻¹ and 880 cm⁻¹ has absorbances that are only due to peracetic acid, and no other component. Thus, this region can be used to quantitatively measure the absorbance of peracetic acid alone, without interferences from the other components of this 4-component system.

As shown in FIG. 1, the PAA absorbance is also present as a triplet of peaks between 920 cm⁻¹ and 970 cm⁻¹. In this region, the absorbance is also only due to peracetic acid, as the other components do not absorb in this band. However, this absorbance is of lower amplitude and has less resolution than the band from 830 cm⁻¹ and 880 cm⁻¹.

The PAA absorbance is also present as a triplet of peaks between 3270 cm⁻¹ and 3330 cm⁻¹ (not shown). In this region, the absorbance is also only due to peracetic acid, as the other components do not absorb in this band.

Likewise, there is another triplet of peaks due to peracetic acid in the MIR region from 1200 cm⁻¹ to 1280 cm⁻¹ as shown in FIG. 1. This triplet PAA band overlaps on one side the band due to hydrogen peroxide, from 1200 cm⁻¹ to 1330 cm⁻¹, especially from 1280 cm⁻¹ to 1330 cm⁻¹. On the other side, the PAA band is overlapped by the acetic acid absorbance bands from 1130 cm⁻¹ to 1220 cm⁻¹, and again from 1250 cm⁻¹ to 1320 cm⁻¹. There is a position in middle peak of this PAA absorbance band that contains a minimal amount of interference from acetic acid and hydrogen peroxide, and can be used for quantitative analysis of the concentration of peracetic acid in the vapor phase.

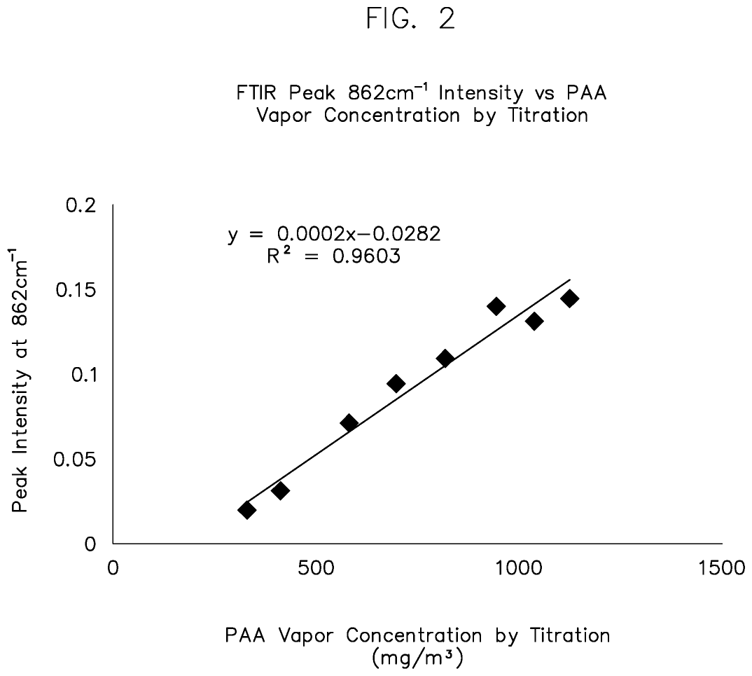

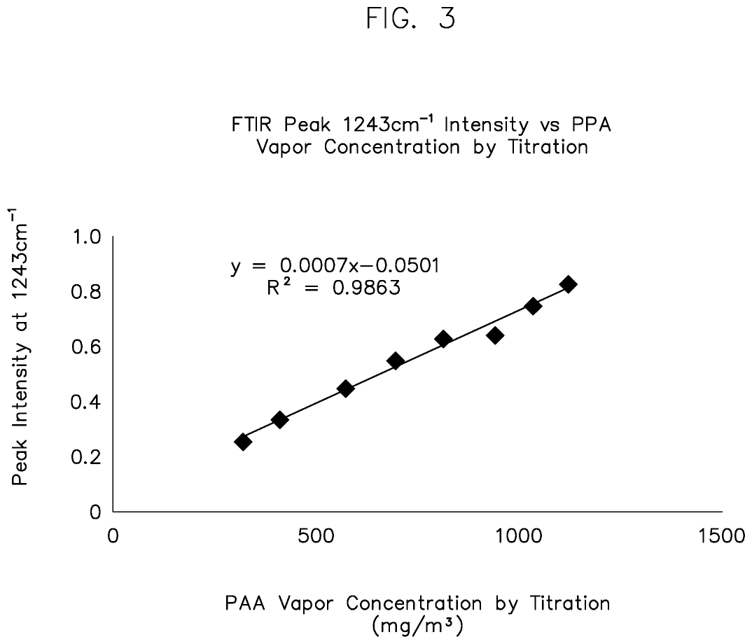

In these bands, the absorbance can be correlated to the concentration, because the vapor absorbance of infrared light obeys Beer's law. The concentration of peracetic acid can thus be shown to be linear and quantitative, for example, as shown in FIGS. 2 and 3.

As shown in FIGS. 1 and 3, the PAA absorbance is linear and can be followed either in the region from 840 cm⁻¹ to 880 cm⁻¹ (FIG. 2), or in the region from 1230 cm⁻¹ to 1250 cm⁻¹ (FIG. 3). PAA absorbance can also be used to calculate PAA concentration in the vapor phase in the region from about 920 cm⁻¹ to about 970 cm⁻¹ and also from about 3270 cm⁻¹ to about 3330 cm⁻¹. Therefore, the absorbance of infrared light in the MIR region is quantitatively related to the concentration of PAA in the vapor phase, by selection of the wavelength range of absorbance.

Additionally, the detection of infrared light in the MIR region between 1220 cm⁻¹ to about 1260 cm⁻¹ yields absorbance data for hydrogen peroxide and peracetic acid. By using chemometrics (since the concentration and the absorbance per mole of the peracetic acid is known) to subtract the contribution of peracetic acid to this region, the vapor phase concentration of hydrogen peroxide is calculated.

The detection of infrared light in the MIR region between 1140 cm⁻¹ to about 1200 cm⁻¹ yields absorbance data for acetic acid.

Thus, using the MIR data, and calculation known to those of skill in the art, the vapor phase concentration of peracetic acid, hydrogen peroxide and acetic acid in the 4-component system can be obtained.

The absorbances due to hydrogen peroxide can be less able to be resolved in the MIR, since the overlap with the peracetic acid peak in the region from 1200 cm⁻¹ to 1260 cm⁻¹ lowers the resolution that is possible with conventional spectroscopic techniques.

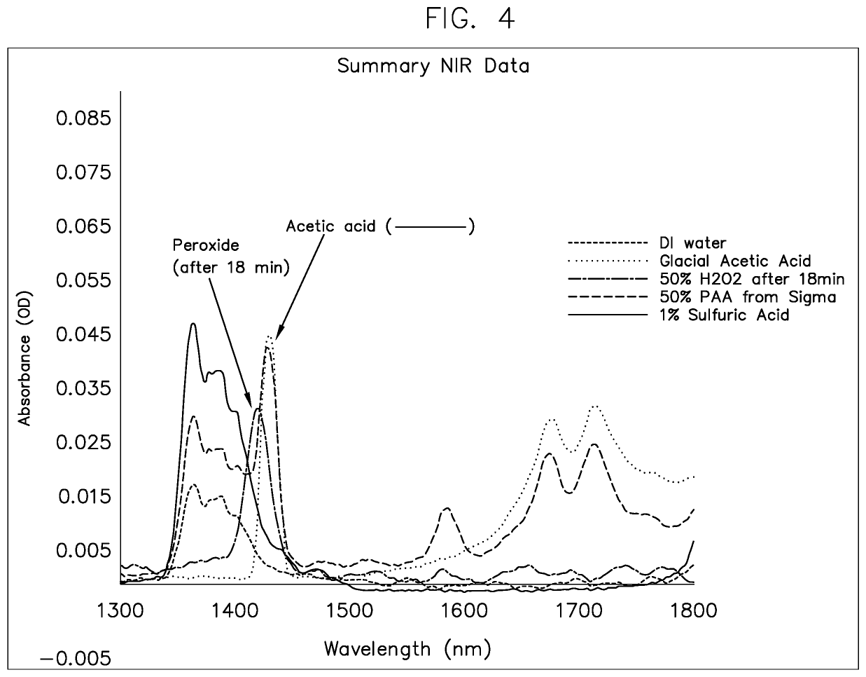

In one embodiment, in order to improve the resolution of the hydrogen peroxide and the acetic acid, the NIR spectrum of the system is used. The NIR spectrum of the system is shown in FIG. 4, for the region between 1300 nm and 1800 nm.

As can be seen in FIG. 4, hydrogen peroxide has an absorbance peak at 1390 nm to 1430 nm. This peak overlaps significantly with the absorbance from acetic acid at 1400 nm to 1450 nm. It must be noted that the 39% PAA from Sigma contained acetic acid, and obscures the resolution of acetic acid from peracetic acid. Ordinarily this region can be used to quantitatively determine acetic acid, if there is no hydrogen peroxide present, or hydrogen peroxide, if there is no acetic acid present. Since this system has both, a measurement of the absorption between 1230 nm and 1450 nm generates a combined hydrogen peroxide+acetic acid concentration. The acetic acid concentration is calculated by measuring the absorption between 1140 cm⁻¹ and 1200 cm⁻¹ in the MIR, and using chemometrics (since both the concentration and the absorbance per mole of the acetic acid is known) to subtract the contribution of acetic acid to this region. The hydrogen peroxide concentration is then calculated.

Thus, using MIR, and optionally NIR data, and some calculation, the vapor phase concentration of peracetic acid, hydrogen peroxide and acetic acid, and peracetic acid in this 4-component system can be individually determined.

The present invention is directed to a peroxy vapor (peroxyacetic acid) and hydrogen peroxide vapor detection system and methods. The system may include (a) a source of peracetic acid vapor, hydrogen peroxide vapor, water vapor and acetic acid vapor, (b) a light source which is configured to supply light with at least a component in the mid-infrared range, and (c) a detector which is configured to individually detect mid-infrared range light in (i) a first mid-infrared spectrum absorbed by the peracetic acid vapor and not absorbed by the hydrogen peroxide vapor, the acetic acid vapor or the water vapor, and (ii) a second mid-infrared spectrum absorbed by the peracetic acid vapor and the hydrogen peroxide vapor.

In one embodiment, a decontamination or sterilization fluid, such as Rapicide PA Sterilant, provided by Medivators (Minneapolis, MN) is utilized. The fluid contains peracetic acid, hydrogen peroxide, acetic acid and water. The fluid may be in liquid form or in vapor form. In an embodiment where the fluid is in liquid form, the liquid is vaporized prior to introduction into the system or method.

The system and method also include a light source which is configured to supply light with at least a component in the mid-infrared range. The light source can be located in the chamber or outside of the chamber. The light source is configured to supply light to the vapor mixture. In one embodiment, the light is in a first mid-infrared spectrum absorbed by the peracetic acid vapor and not absorbed by the hydrogen peroxide vapor, the acetic acid vapor or the water vapor, for example, from about 920 cm⁻¹ to about 970 ⁻¹, from about 830 cm⁻¹ to about 880 cm⁻¹, or from about 1220 cm⁻¹ to about 1260 cm⁻¹. In another embodiment, the light is in a second mid-infrared spectrum absorbed by the peracetic acid vapor and the hydrogen peroxide vapor, such as from about 1220 cm⁻¹ to about 1260 cm⁻¹. In another embodiment, the light is in a third mid-infrared spectrum absorbed by the acetic acid vapor, for example, from about 1140 cm⁻¹ to about 1200 cm⁻¹. In another embodiment, the light is in the near infrared spectrum. The light in the near infrared spectrum can be absorbed by the peracetic acid vapor, the hydrogen peroxide vapor and the acetic acid vapor, for example, from about 1390 nm to about 1430 nm.

In one embodiment, the light source is a single light source that supplies the light in the mid-infrared spectrum. In another embodiment, the system or method utilizes multiple light sources to supply light in narrow ranges in the mid infrared spectrum. In another embodiment, a separate light source supplies light in the near infrared spectrum.

In one embodiment, the light source supplies light to the vapor mixture prior to a disinfection or sterilization step. In another embodiment, the light source supplies light to the vapor mixture during a disinfection or sterilization step. In another embodiment, the light source supplies light to the vapor mixture after a disinfection or sterilization step. In another embodiment, the light source supplies light at various times during the process.

In one embodiment, the light is supplied into the chamber. In another embodiment, the vapor mixture is sampled and placed into a gas cell, where the light is supplied.

The system and method of the present invention also include a detector which is configured to individually detect mid-infrared range light. In one embodiment, the detector detects light in a first mid-infrared spectrum absorbed by the peracetic acid vapor and not absorbed by the hydrogen peroxide vapor, the acetic acid vapor or the water vapor, for example, from about 920 cm⁻¹ to about 970 cm⁻¹, from about 830 cm⁻¹ to about 880 cm⁻¹, or from about 1220 cm⁻¹ to about 1260 cm⁻¹. In another embodiment, the detector detects light in a second mid-infrared spectrum absorbed by the peracetic acid vapor and the hydrogen peroxide vapor, such as from about 1220 cm⁻¹ to about 1260 cm⁻¹. In another embodiment, the detector detects light in a third mid-infrared spectrum absorbed by the acetic acid vapor, for example, from about 1140 cm⁻¹ to about 1200 cm⁻¹. In another embodiment, the detector detects light in the near infrared spectrum. The light in the near infrared spectrum can be absorbed by the peracetic acid vapor, the hydrogen peroxide vapor and the acetic acid vapor, for example, from about 1390 nm to about 1430 nm.

In one embodiment, the detector which detects mid-infrared light and the detector which detects near-infrared light are a single detector. In another embodiment, the detector which detects mid-infrared light and the detector which detects near-infrared light are separate detectors.

The detector can be located in the chamber or outside of the chamber. In one embodiment, the vapor mixture can be pulled or sampled from the chamber and analyzed. In another embodiment, the detector can be placed in-line on a scope flow channel to analyze gas coming through the scope from inside the chamber.

In one embodiment, the systems and methods of the present invention may also include a processor. The processor is configured to determine at least a concentration of the peracetic acid vapor from the detected light in the first mid-infrared spectrum. In another embodiment the processor can also determine a concentration of the hydrogen peroxide, and/or acetic acid vapor. The processor can be configured to calculate the concentrations from the detected light in the MIR range as well as the NIR range.

In one embodiment, the processor is configured to determine at least one of (a) an absorbance of light in the first mid-infrared spectrum and (b) a transmittance of light in the first mid-infrared spectrum, and is further configured to convert the determined absorbance or transmittance into the concentration of the peracetic acid vapor.

In another embodiment the processor is configured to determine at least one of (a) an absorbance of light in the second mid-infrared spectrum and (b) a transmittance of light in the second mid-infrared spectrum, and to convert the determined absorbance or transmittance into a concentration of the hydrogen peroxide vapor.

In another embodiment, the processor is configured to determine at least one of (a) an absorbance of light in the third mid-infrared spectrum and (b) a transmittance of light in the third mid-infrared spectrum, and to convert the determined absorbance or transmittance into a concentration of the acetic acid vapor

In another embodiment, the processor is configured to determine at least one of (a) an absorbance of light in the near-infrared spectrum and (b) a transmittance of light in the near-infrared spectrum, and to convert the determined absorbance or transmittance into a concentration of the hydrogen peroxide vapor.

In one embodiment, the IR absorbance of PAA is calculated as follows:
1—Determine the IR signal of PAA at 860 wavenumbers
2—Determine the IR signal of the background IR absorbance at 820 wavenumbers.
3—Subtract the background signal at 820 from the PAA signal at 860. Eg: PAA signal=(signal at 860)−(signal at 820)

In one embodiment, the IR absorbance of H₂O₂ is calculated as follows:
1—Determine the combined signal of PAA+H₂0₂ at 1250 wavenumbers
2—Determine the signal of PAA at 860 wavenumbers
3—Determine the background signal at 820 wavenumbers or at 1115 wavenumbers.
4—Using a PAA (TAED chemistry) only solution, determine the ratio of the PAA peak at 1250 wavenumbers to 860 wavenumbers. E.g. Ratio=(PAA signal at 1250)/(PAA signal at 860).
5—Determine the PAA signal at 1250 wavenumbers by multiplying the PAA signal at 860 by the ratio determined dently in step 4.
6—To Determine H₂O₂ at 1250 wavenumbers: from the signal determined in Step 1, subtract the PAA signal determined in step 5 and the signal determined in step 3.

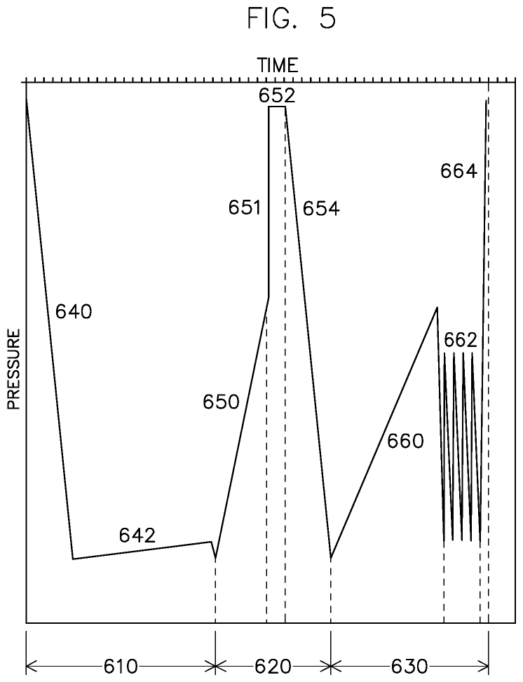

FIG. 5 shows a graph of pressure versus time within an exemplary decontamination or sterilization chamber in an example embodiment of a decontamination or sterilization cycle. As shown in FIG. 5, the X-axis of the graph illustrates time or duration, and the Y-Axis illustrates pressure within the decontamination chamber. As shown in FIG. 5, in some embodiments, an exemplary cycle may include multiple pressure changes within the chamber. The cycle or a portion of the cycle illustrated in FIG. 5 may be repeated several times within a decontamination or sterilization process.

The cycle of FIG. 5 includes a vacuum preconditioning step 610, a first decontamination or sterilization step 620, and a second decontamination or sterilization step 630. The vacuum preconditioning step 610 includes a first pump down 640 in which pressure is drawn from the chamber and an optional lumen warm up period 642. During the lumen warm up period 642, the pressure within the chamber is held relatively steady.

In some embodiments, the vacuum preconditioning step 610 may be followed by the first decontamination or sterilization step 620. During the first decontamination or sterilization step 620, the vapor mixture is injected into the chamber in a first injection step 650. During the first injection step 650 the pressure within the chamber increases. In an example embodiment, the vapor mixture is injected into the decontamination chamber during the first injection step 650. The vapor mixture may be injected into the chamber at a single injection at a constant rate as shown in the first injection step 650 or it may be injected in a plurality of stepwise injections.

The first injection step 650 may be optionally followed by a pressure increase step 651. During the pressure increase step 651, the pressure inside the chamber is increased to a suitable pressure determined to increase the effectiveness of a decontamination or sterilization process. After the vapor mixture is injected, it may be optionally allowed to diffuse throughout the chamber in a diffusion period 652 while the pressure is held steady. In some embodiments, the optional diffusion period 652 is not used.

In some embodiments, after the diffusion period 652, a second pump down 654 may be carried out. During the second pump down 654, the pressure within the chamber decreases. The second decontamination or sterilization step 630 is carried out after the second pump down 654. During the second decontamination or sterilization step 630, a second injection step 660 may be used to add the vapor mixture to the decontamination chamber while the pressure within the chamber increases. The second injection step 660 may include adding the vapor mixture into the decontamination chamber in a single injection step or in a plurality of stepwise injection steps that may be used to gradually add the vapor mixture to the chamber.

In some embodiments, a pump may be used to direct air within the chamber through the lumen or lumens of the device in coordination with the cycle. For example, during the first injection step 650, the second injection step 660 or both injection steps, a pump may be used to direct air within the chamber towards and/or through the lumens of the device. In some embodiments, the pump may be turned on before or during either the first or second injection step 650, 660. For example, the pump may be turned on with or substantially with the first and/or second injection steps 650, 660. In some embodiments, the pump may turn on before or during the first injection step 650 and may turn off at the end of or after the first injection step 650. Additionally or alternatively, the pump may turn on before or during the second injection step 660 and may turn off after or at the end of the second injection step 660. In some embodiments, the pump may turn on before or during both the first and second injection steps 650, 660, or the pump may be turned on before or at the beginning of the first injection step 650 and may be turned off during or after the end of the second injection step 660.

After the second injection step 660, a plurality of air washes 662 may be carried out. As shown in FIG. 5, the plurality of air washes 662 may include increasing and decreasing the pressure within the chamber repeatedly. In some embodiments, the pump may be run during the plurality of air washes 662 to force air along the inside of the device to be decontaminated or sterilized. The air washes may be carried any number of times to remove a suitable amount of vapor mixture from the chamber. After a suitable number of air washes 662, the pressure within the chamber may be allowed to reach atmospheric pressure in a final vent step 664.

Illumination and detection of the vapor mixture can occur at any point in the process. In one embodiment, the vapor mixture is analyzed throughout the process.

The following paragraphs provide for various aspects of the present invention.

In one embodiment, in a first paragraph (1), the present invention provides a peracetic acid vapor and hydrogen peroxide vapor detection system, the system comprising a source of peracetic acid vapor, hydrogen peroxide vapor, water vapor and acetic acid vapor: a light source configured to supply light with at least a component in the mid-infrared range; and a detector configured to individually detect mid-infrared range light in (a) a first mid-infrared spectrum absorbed by the peracetic acid vapor and not absorbed by the hydrogen peroxide vapor, the acetic acid vapor or the water vapor, and (b) a second mid-infrared spectrum absorbed by the peracetic acid vapor and the hydrogen peroxide vapor.
2. The system of paragraph 1, wherein the first mid-infrared spectrum is from about 920 cm⁻¹ to about 970 cm⁻¹.
3. The system of paragraph 1, wherein the first mid-infrared spectrum is from about 830 cm⁻¹ to about 880 cm⁻¹.
4. The system of paragraph 1, wherein the first mid-infrared spectrum is from about 3270 cm⁻¹ to about 3330 cm⁻¹.
5. The system of any of paragraphs 1 through 4, wherein the second mid-infrared spectrum is from about 1220 cm⁻¹ to about 1260 cm⁻¹.
6. The system of any of paragraphs 1 through 5, wherein the detector further individually detects a third mid-infrared spectrum absorbed by the acetic acid vapor.
7. The system of paragraph 6, wherein the third mid-infrared spectrum is from about 1140 cm⁻¹ to about 1200 cm⁻¹.
8. The system of any of paragraphs 1 through 7, wherein the light source is a single light source that supplies the light in the first and second mid-infrared spectrum.
9. The system of paragraph 8, wherein the single light source supplies light in the third mid-infrared spectrum.
10. The system of any of paragraphs 1 through 7, wherein the light source is a pair of light sources, wherein a first light source supplies the light in the first mid-infrared spectrum and a second light source supplies the light in the second mid-infrared spectrum.
11. The system of claim 10, wherein the light source further comprises a third light source that supplies the light in the third mid-infrared spectrum.
12. The system of any of paragraphs 1 through 11, further comprising a light source which supplies light with at least a component in the near-infrared range.
13. The system of paragraph 12, further comprising a detector which individually detects near-infrared range light in a near-infrared spectrum absorbed by the peracetic acid vapor, the hydrogen peroxide vapor and the acetic acid vapor.
14. The system of paragraph 13, wherein the detector which detects mid-infrared light and the detector which detects near-infrared light are a single detector.
15. The system of paragraph 13, wherein the detector which detects mid-infrared light and the detector which detects near-infrared light are separate detectors.
16. The system of any of paragraphs 13 through 15, wherein the near-infrared spectrum is from about 1390 nm to about 1430 nm.
17. The system of any of paragraphs 1 through 16, further comprising; a processor configured to determine at least a concentration of the peracetic acid vapor from the detected light in the first mid-infrared spectrum.
18. The system of paragraph 17, wherein the processor is configured to determine at least a concentration of the hydrogen peroxide vapor from the detected light in the second mid-infrared range spectrum.
19. The system of paragraph 17, wherein the processor is configured to determine at least a concentration of the hydrogen peroxide vapor from the detected light in the near-infrared spectrum.
20. The system of any of paragraphs 17 through 19, wherein the processor is configured to determine at least a concentration of the acetic acid vapor from the detected light in the third mid-infrared range spectrum.
21. The system of any of paragraphs 17 through 20, wherein the processor is configured to determine at least one of (a) an absorbance of light in the first mid-infrared spectrum and (b) a transmittance of light in the first mid-infrared spectrum, and is further configured to convert the determined absorbance or transmittance into the concentration of the peracetic acid vapor.
22. The system of any of paragraphs 17 through 21, wherein the processor is further configured to determine at least one of (a) an absorbance of light in the second mid-infrared spectrum and (b) a transmittance of light in the second mid-infrared spectrum, and to convert the determined absorbance or transmittance into a concentration of the hydrogen peroxide vapor.
23. The system of any of paragraphs 17 through 21, wherein the processor is further configured to determine at least one of (a) an absorbance of light in the near-infrared spectrum and (b) a transmittance of light in the near-infrared spectrum, and to convert the determined absorbance or transmittance into a concentration of the hydrogen peroxide vapor.
24. The system of any of paragraphs 21 through 23, wherein the processor is further configured to determine at least one of (a) an absorbance of light in the third mid-infrared spectrum and (b) a transmittance of light in the third mid-infrared spectrum, and to convert the determined absorbance or transmittance into a concentration of the acetic acid vapor.
25. The system of any of paragraphs 1 through 24, further comprising a source of a liquid peracetic acid, hydrogen peroxide, acetic acid and water mixture and a vaporizer for vaporizing the liquid mixture to form the peracetic acid vapor, the hydrogen peroxide vapor, the acetic acid vapor and the water vapor.
26. A peracetic acid and hydrogen peroxide treatment system comprising: a treatment chamber;
   a vaporizer configured for generating a mixture of peracetic acid vapor, hydrogen peroxide vapor, water vapor and acetic acid vapor and supplying the vapor mixture to the treatment chamber;
   a light source configured to supply light with at least a component in the mid-infrared range;
   a detector configured to individually detect mid-infrared range light in a first spectrum absorbed by peracetic acid vapor and not any of the hydrogen peroxide vapor, water vapor and acetic acid vapor, and a second spectrum absorbed by the peracetic acid vapor and the hydrogen peroxide vapor; and,
   a processor configured to determine the concentration of the peracetic acid vapor in the treatment chamber.
27. The system of paragraph 26, wherein the treatment is sterilization.
28. The system of paragraph 26, wherein the treatment is disinfection.
29. The system of any of paragraphs 26 through 28, wherein the processor is further configured to determine the concentration of the hydrogen peroxide vapor in the treatment chamber.
30. The system of any of paragraphs 26 through 29, wherein the first mid-infrared spectrum is from about 920 cm⁻¹ to about 970 cm⁻¹.
31. The system of any of paragraphs 26 through 29, wherein the first mid-infrared spectrum is from about 830 cm⁻¹ to about 880 cm⁻¹.
32. The system of any of paragraphs 26 through 29, wherein the first mid-infrared spectrum is from about 3270 cm⁻¹ to about 3330 cm⁻¹.
33. The system of any of paragraphs 26 through 32, wherein the second mid-infrared spectrum is from about 1220 cm⁻¹ to about 1260 cm⁻¹.
34. The system of any of paragraphs 26 through 33, wherein the detector further individually detects a third mid-infrared spectrum absorbed by the acetic acid vapor.
35. The system of paragraph 34, wherein the processor is further configured to determine the concentration of the acetic acid vapor in the treatment chamber.
36. The system of either of paragraphs 34 or 35, wherein the third mid-infrared spectrum is from about 1140 cm⁻¹ to about 1200 cm⁻¹.
37. The system of any of paragraphs 26 through 36, wherein the light source is a single light source that supplies the light in the first and second mid-infrared spectrum.
38. The system of paragraph 37, wherein the single light source supplies light in the third mid-infrared spectrum.
39. The system of any of paragraphs 26 through 36, wherein the light source is a pair of light sources, wherein a first light source supplies the light in the first mid-infrared spectrum and a second light source supplies the light in the second mid-infrared spectrum.
40. The system of paragraph 39, wherein the light source further comprises a third light source that supplies the light in the third mid-infrared spectrum.
41. The system of any of paragraphs 26 through 40, further comprising a light source which supplies light with at least a component in the near-infrared range.
42. The system of paragraph 41, further comprising a detector which individually detects near-infrared range light in a near-infrared spectrum absorbed by the hydrogen peroxide vapor and the acetic acid vapor.
43. The system of paragraph 42, wherein the detector which detects mid-infrared light and the detector which detects near-infrared light are a single detector.
44. The system of paragraph 42, wherein the detector which detects mid-infrared light and the detector which detects near-infrared light are separate detectors.
45. The system of any of paragraphs 41 through 44, wherein the wherein the near-infrared spectrum is from about 1390 nm to about 1430 nm.
46. The system of any of paragraphs 41 through 45, wherein the processor is configured to determine at least a concentration of the hydrogen peroxide vapor from the detected light in the near-infrared spectrum.
47. The system of any of paragraphs 26 through 46, wherein the processor is configured to determine at least one of (a) an absorbance of light in the first mid-infrared spectrum and (b) a transmittance of light in the first mid-infrared spectrum, and is further configured to convert the determined absorbance or transmittance into the concentration of the peracetic acid vapor.
48. The system of paragraph 47, wherein the processor is further configured to determine at least one of (a) an absorbance of light in the second mid-infrared spectrum and (b) a transmittance of light in the second mid-infrared spectrum, and to convert the determined absorbance or transmittance into the concentration of the hydrogen peroxide vapor.
49. The system of paragraph 47, wherein the processor is further configured to determine at least one of (a) an absorbance of light in the near-infrared spectrum and (b) a transmittance of light in the near-infrared spectrum, and to convert the determined absorbance or transmittance into a concentration of the hydrogen peroxide vapor.
50. The system of any of paragraphs 47 through 49, wherein the processor is further configured to determine at least one of (a) an absorbance of light in the third mid-infrared spectrum and (b) a transmittance of light in the third mid-infrared spectrum, and to convert the determined absorbance or transmittance into a concentration of the acetic acid vapor.
51. The system of any of paragraphs 26 through 50, wherein the system is configured to treat a medical device.
52. The system of paragraph 51, wherein the medical device is an endoscope.
53. A disinfection or sterilization system comprising:
   (a) a treatment chamber;
   (b) a vaporizer configured to vaporize an aqueous solution comprising peracetic acid, hydrogen peroxide, acetic acid and water to form a mixture of peracetic acid vapor, a hydrogen peroxide vapor, an acetic acid vapor and a water vapor and for supplying the mixture of vapors to the treatment chamber;
   (c) a light source configured to project a beam of light in a mid-infrared range through the mixture of vapors;
   (d) a mid-infrared light detector configured to detect a first spectrum absorbed by the peracetic acid vapor and not any of the hydrogen peroxide vapor, the acetic acid vapor and the water vapor, and a second spectrum absorbed by the peracetic acid vapor and the hydrogen peroxide vapor;
   (e) a first processor configured to convert the detected first and second spectrum light into one of (a) absorbance values indicative of mid infrared light absorbed by the peracetic acid and hydrogen peroxide vapors and (b) transmittance values indicative of mid-infrared light transmitted through the peracetic acid and hydrogen peroxide vapors; and
   (f) a second processor configured to convert the determined absorbance or transmittance values into a concentration of the peracetic acid vapor and a concentration of the hydrogen peroxide vapor.
54. The system of paragraph 53, wherein the first mid-infrared spectrum is from about 920 cm⁻¹ to about 970 cm⁻¹.
55. The system of paragraph 53, wherein the first mid-infrared spectrum is from about 830 cm⁻¹ to about 880 cm⁻¹.
56. The system of paragraph 53, wherein the first mid-infrared spectrum is from about 3270 cm⁻¹ to about 3330 cm⁻¹.
57. The system of any of paragraphs 53 through 56, wherein the second mid-infrared spectrum is from about 1220 cm⁻¹ to about 1260 cm⁻¹.
58. The system of any of paragraphs 53 through 57, wherein the detector further individually detects a third mid-infrared spectrum absorbed by the acetic acid vapor.
59. The system of paragraph 58, wherein the third mid-infrared spectrum is from about 1140 cm⁻¹ to about 1200 cm⁻¹.
60. The system of any of paragraphs 53 through 59, wherein the light source is a single light source that supplies the light in the first and second mid-infrared spectrum.
61. The system of paragraph 60, wherein the single light source supplies light in the third mid-infrared spectrum.
62. The system of any of paragraphs 53 through 59, wherein the light source is a pair of light sources, wherein a first light source supplies the light in the first mid-infrared spectrum and a second light source supplies the light in the second mid-infrared spectrum.
63. The system of paragraph 62, wherein the light source further comprises a third light source that supplies the light in the third mid-infrared spectrum.
64. The system of any of paragraphs 53 through 63, further comprising a light source which supplies light with at least a component in the near-infrared range.
65. The system of paragraph 64, further comprising a detector which individually detects near-infrared range light in a near-infrared spectrum absorbed by the hydrogen peroxide vapor and the acetic acid vapor.
66. The system of paragraph 65, wherein the detector which detects mid-infrared light and the detector which detects near-infrared light are a single detector.
67. The system of paragraph 65, wherein the detector which detects mid-infrared light and the detector which detects near-infrared light are separate detectors.
68. The system of any of paragraphs 65 through 67, wherein the wherein the near-infrared spectrum is from about 1390 nm to about 1430 nm.
69. The system of any of paragraphs 65 through 68, wherein the first processor is further configured to convert the detected near-infrared light into one of (a) absorbance values indicative of near-infrared light absorbed by the hydrogen peroxide vapor and the acetic acid vapor and (b) transmittance values indicative of near-infrared light transmitted through the hydrogen peroxide vapor and the acetic acid vapor; and the second processor is further configured to convert the determined absorbance or transmittance values into a concentration of the hydrogen peroxide vapor or acetic acid vapor.
70. The system of any of paragraphs 58 through 68, wherein the first processor is further configured to determine at least one of (a) an absorbance of light in the third mid-infrared spectrum and (b) a transmittance of light in the third mid-infrared spectrum, and the second processor is further configured to convert the determined absorbance or transmittance into a concentration of the acetic acid vapor.
71. The system of any of paragraphs 53 through 70 wherein the first and second processors are a single processor.
72. The system of any of paragraphs 53 through 70 wherein the first and second processors are separate processors.
73. The system of any of paragraphs 53 through 72, wherein the system is configured to disinfect or sterilize a medical device.
74. The system of paragraph 73, wherein the medical device is an endoscope.
75. A method for detecting the presence of peracetic acid and hydrogen peroxide in a vapor mixture, comprising the steps of
   providing a vaporized mixture comprising peracetic acid, hydrogen peroxide, acetic acid, and water into a chamber;
   projecting light in a mid-infrared range through a portion of the vaporized mixture that has passed through at least a portion of the chamber;
   detecting mid-infrared light in a first spectrum absorbed by the peracetic acid vapor and not any of the hydrogen peroxide vapor, the acetic acid vapor and the water vapor, and a second narrow spectrum absorbed by a peracetic acid vapor and hydrogen peroxide vapor; and
   detecting mid-infrared light in a second spectrum absorbed by the peracetic acid vapor and the hydrogen peroxide vapor.
76. The method of paragraph 75, further comprising determining at least a concentration of the peracetic acid vapor from the light detected in first spectrum.
77. The method of either of paragraphs 75 or 76, further comprising determining a concentration of the hydrogen peroxide from the light detected in the second narrow spectrum.
78. The method of any of paragraphs 75 through 77, wherein the first mid-infrared spectrum is from about 920 cm⁻¹ to about 970 cm⁻¹.
79. The method of any of paragraphs 75 through 77, wherein the first mid-infrared spectrum is from about 830 cm⁻¹ to about 880 cm⁻¹.
80. The method of any of paragraphs 75 through 77, wherein the first mid-infrared spectrum is from about 3270 cm⁻¹ to about 3330 cm⁻¹.
81. The method of any of paragraphs 75 through 80, wherein the second mid-infrared spectrum is from about 1220 cm⁻¹ to about 1260 cm⁻¹.
82. The method of any of paragraphs 75 through 81, further comprising the step of detecting mid-infrared light in a third spectrum absorbed by the acetic acid vapor.
83. The method of paragraph 82, further comprising determining at least a concentration of the acetic acid vapor from the light detected in third spectrum.
84. The method of either of paragraphs 82 or 83, wherein the third mid-infrared spectrum is from about 1140 cm⁻¹ to about 1200 cm⁻¹.
85. The method of any of paragraphs 75 through 84, wherein the vaporized mixture is provided at a pressure of less than 650 torr.
86. The method of any of paragraphs 75 through 85, wherein detecting mid-infrared light in the first spectrum and detecting mid-infrared light in the second spectrum are carried out sequentially.
87. The method of any of paragraphs 75 through 85, wherein detecting mid-infrared light in the first spectrum and detecting mid-infrared light in the second spectrum are carried out in parallel.
88. The method of paragraph 87, wherein
   (a) mid-infrared light in the first spectrum is detected at a pressure less than 200 torr;
   (b) the pressure is set to greater than 650 torr for a period of time after detecting mid-infrared light in the first spectrum,
   (c) the pressure is thereafter reduced to less than 200 torr after the period of time; and the mid-infrared light in the second spectrum is detected.
89. The method of any of paragraphs 75 through 88, further comprising converting the determined spectrums into one of absorbance and transmittance of mid infrared light through the vapor mixture and converting the determined one of the absorbance and transmittance into the concentration of the peracetic acid vapor and a concentration of the hydrogen peroxide vapor.
90. The method of any of paragraph 75 through 89, wherein the method is carried out in a system of any of paragraphs 1 through 75.
91. A method for detecting the presence of peracetic acid and hydrogen peroxide
   (a) in a vapor mixture, comprising the steps of
   (b) providing a vaporized mixture comprising peracetic acid, hydrogen peroxide, acetic acid, and water into a chamber;
   (c) projecting light in a mid-infrared range through a portion of the vapor mixture that has passed through a portion of the chamber;
   (d) detecting mid-infrared light in a first spectrum absorbed by the peracetic acid vapor and not any of the hydrogen peroxide vapor, the acetic acid vapor and the water vapor, and a second narrow spectrum absorbed by a peracetic acid vapor and hydrogen peroxide vapor;
   (e) projecting light in a near-infrared range through the monitored region of the chamber; and
   (f) detecting near-infrared light in a spectrum absorbed by the peracetic acid vapor, the hydrogen peroxide vapor and the acetic acid vapor.
92. The method of paragraph 91, wherein the first mid-infrared spectrum is from about 920 cm⁻¹ to about 970 cm⁻¹.
93. The method of paragraph 91, wherein the first mid-infrared spectrum is from about 830 cm⁻¹ to about 880 cm⁻¹.
94. The method of paragraph 91, wherein the first mid-infrared spectrum is from about 3270 cm⁻¹ to about 3330 cm⁻¹.
95. The method of any of paragraphs 91 through 94, wherein the near-infrared spectrum is from about 1390 nm to about 1430 nm.
96. The method of any of paragraphs 91 through 95, further comprising determining at least a concentration of the peracetic acid vapor from the light detected in the first mid-infrared spectrum.
97. The method of any of paragraphs 91 through 96, further comprising determining at least a concentration of the hydrogen peroxide vapor from the light detected the near-infrared spectrum.
98. The method of any of paragraphs 91 through 97, further comprising detecting mid-infrared light in a third spectrum absorbed by the acetic acid vapor.
99. The method of paragraph 98, wherein the third mid-infrared spectrum is from about 1140 cm⁻¹ to about 1200 cm⁻¹.
100. The method of either of paragraphs 98 or 99, further comprising determining at least a concentration of the acetic acid vapor from the light detected in the third mid-infrared spectrum.
101. The method of any of paragraphs 91 through 100, carried out in a system of any of paragraphs 1 through 75.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

The following examples illustrate the principles and advantages of the invention.

### EXAMPLES

### Example 1

Low temperature sterilization system (LTSS) with IR sampler

A Revox low temperature sterilization system (LTSS) model number 5434 (Serial number RVXM5434) was configured with an experimental infrared vapor detector (EVD) inline to a selected scope flow channel. The EVD was programed to capture a 6-scan-averaged infrared spectrum from 1300 to 800 cm⁻¹ every ten seconds using a resolution of 4 cm⁻¹. Infrared data collection was set to begin the moment the LTSS cycle was started.

The LTSS was configured to deliver 5.0 mL of vaporized Revox PA sterilant (peracetic acid, hydrogen peroxide, acetic acid and water from Medivators) into a 417-liter vacuum chamber after the vacuum pressure had reached 10 torr. Sterilant injection resulted in a final system pressure of 150 torr at which point the system's scope flow channels were turned on to allow chamber gasses to pass through the EVD. Scope flow was allowed to continue for 900 seconds and the system was then ventilated using a 4 cycle ventilation process. Infrared data collection was set to stop once the ventilation cycles were completed.

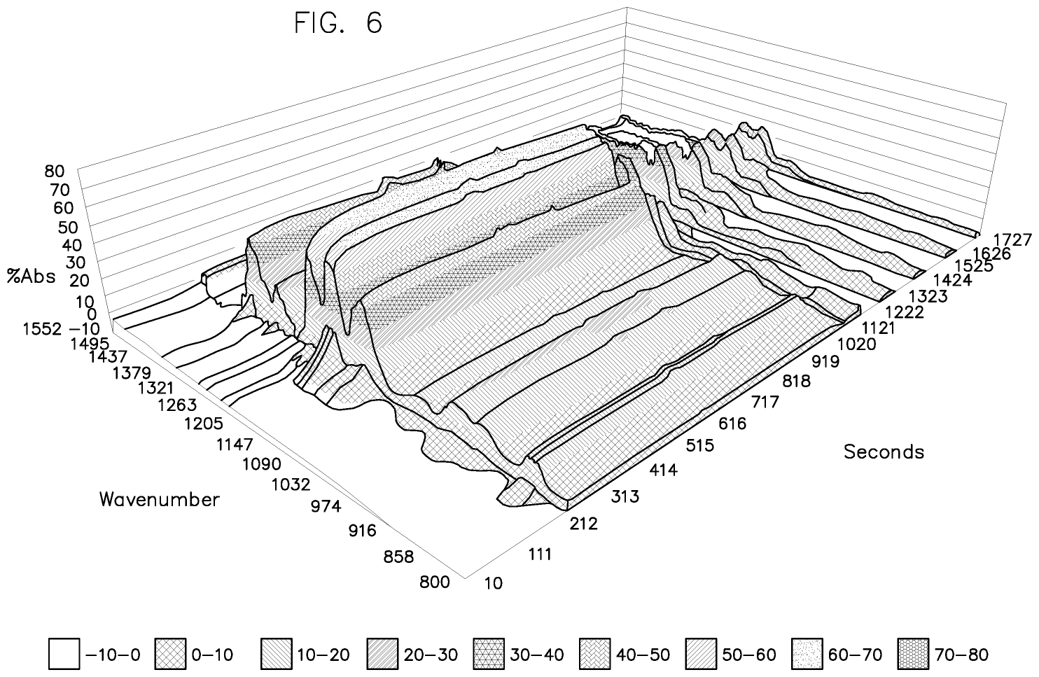

Spectra signals were monitored in time and are shown in FIG. 6. The 3 dimensional spectra shows time (z axis) at the given wavenumbers (x axis) for the chemical of interest. Increasing % Absorbance (y axis) is directly proportional to the concentration. Once the % absorbance crosses a given threshold, it is considered to have a high enough concentration of PAA to cause sterilization.

### Example 2

The experimental setup included three key components, 1) the Thermo Nicolet 380 FTIR system running the Thermo Omnic Software, 2) the Nicolet 2 meter gas accessory, and 3) a Thermo MCTA liquid nitrogen detector. Gas analysis was performed by connecting the main large sterilization chamber to the 2 m gas cell so that all gas species could be detected/monitored in real time during the entire sterilization cycle. A Thermo Nicolet Avatar 380 FTIR was used in its standard configuration (with a KBR beamsplitter) and an MCTA detector. The MCTA detector is preferred since it provides 10×higher sensitivity compared with standard room temperature DTGS detector. Long path length cells (2 m and higher) intrinsically lose light due their long path length, thus making the MCTA detector a better option. During the entire cycle, data points were taken every 10 s (with averaging) to get repeatable results with high signal to noise ratios.

The Thermo Avatar 380 FTIR had a minimum resolution of 4 cm⁻¹, utilizing a 24 bit A/D, USB 2.0, a Mid IR source, and a resolution of 4 wavenumbers and a scan speed of 6 scans per data point. all done in absorbance mode. The background was taken and stored just prior to introducing the liquids in vacuum. Automatic logging was used in Omnic to store each trace in SPA file format every 10 s. The Mid Infrared spectral range for the enitre system was 7000-650 cm⁻¹. The 2 m gas cell had a volume of 200 mL with detection limits capable of 50-200 ppb. The smaller size of the 2 m cell is preferred over larger cells (10 m) for their low sample volumes to more accurately monitor changes in the kinetics. The Nicolet MCTA detector had the following specifications: 11700-600 cm⁻¹, detector area: 1×1 mm\hat{ }2, D*: 4.7 e\hat{ }10 cm Hz\hat{ }1/2 W⁻¹, response: 750 V/W, bandwidth: of 175 Hz.

### Example 3

The purpose of this example was to find a correlation of the FTIR absorbance of peracetic acid vapor and the concentration of peracetic acid vapor (mg/L) during the sterilization cycle by running three separate runs and generating a calibration curve. This example also shows how to find the calibration curve for peracetic acid vapor (mg/L) with peracetic acid IR absorption.

Material
Agilent pump, Model: G1310B 1260 IsoPump, SN: DEAB903915
Ivek nozzle (long nozzle, Sonicair 5 mm, Ext Tip, Encap 0.5 mm PN 14658⁻¹5)
Magtech % RH and Temperature sensor
Chemistry: an aqueous solution comprising approximately 5% PAA, 23% H₂0₂, 6% AA and the remainder water.
Balance, OHAUS Adventurer, SN: C5954
Vaporization chamber test bed with 120 L chamber
Timer
FTIR Detection Experiment:
   FTIR system, Thermo, Nicolet iS5, SN: ASB1817658
   10-meter gas cell
   PTFE tubing
FTIR program configuration:
   Number of Scans: 4
   Resolution: 4
   Absorbance mode
   Sample Compartment: Main
   Detector: DGTS KBr
   Beam Splitter: KBr
   Source: IR
   Accessory: iD Base Adaptet Plate for Nicolet iS5
   Window: ZnSe
   Range: 1600-800 cm⁻¹
   Gain: 8
   Optical Velocity: 0.4747
   Aperture: 100

Procedure:

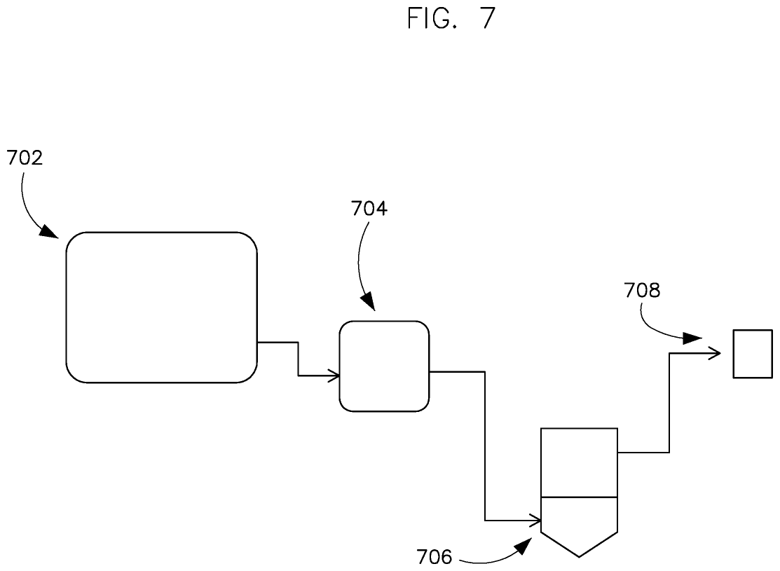

The setup of sampling collection is shown in FIG. 7.

Before each run, the RH and temperature sensors were placed into the chamber 702. The test cycle was started by evacuating the chamber 702 down to 10 torr. At 10 torr, the injection was started. The injection volumes tested are shown in Table 1 with the liquid flow rate and air flow rate used for the injection process.

TABLE 1
Injection process condition used.

| Injection | Chemistry flowrate | Injection Air flow |
|---|---|---|
| Vol (mL) | (mL/min) | rate (L/min) |
| 0.5 | 2 | 3.5 |
| 1.0 | 2 | 3.5 |
| 1.5 | 2 | 3.5 |
| 2.5 | 2 | 3.5 |
| 3.0 | 2 | 3.5 |
| 3.5 | 2 | 3.5 |
| 4.0 | 2 | 3.5 |

Once the set volume of chemistry had been injected (based on the mass reading from the scale using density of 1.18 g/mL), the liquid injection was stopped and the air injection continued until the chamber attained operation pressure (100 torr for Tables 2 and 3 or 75 torr for Table 4).

Immediately after the injections, the vacuum pump 708 was turned on and FTIR chemistry sampling 704 and cold trap sampling 706 were collected for 5 min (for 0.5-3mL injection) and 3 min (for 3.5-4mL injection) after the injection process was completed. The pressure of the chamber was recorded with each sampling time (before and after the sampling process).

After the sampling process was completed, the venting process was initiated. The chamber was vented with 4 venting cycle to remove the chemistry from the chamber.

IR analysis: For all IR absorbance data, the absorbance value obtained was defined as the highest absorbance observed at 860 cm⁻¹ during the vapor sample process compared to a baseline reading which was taken at 820 cm⁻¹.

Cold trap sample analysis: after leaving the IR chamber 704, the gas was collected in 10 mL of water via a cold trap 706. An HPLC method for organic acids was used to analyze the total mg of vapor (H₂O₂, PAA, and acetic acid) in the collected sample.

A correlation curve between cold trap PAA vapor (mg/L) data with PAA-IR absorbance was then made.

Results:

The calculation of vapor concentration in mg/L, were done based on the Ideal Gas law.
Initial Pressure: Pressure of the chamber immediately prior to taking sample=P₁
Final Pressure: Pressure of the chamber immediately after taking sample=P₂
Temp.(T): Average Temperature of chamber during sampling.
Initial total mole (n₁): Total mole of gases (Air, H₂O₂, PAA, AA and water) in the chamber before sampling.
Final total mole (n₂): Total mole of gases (Air, H₂O₂, PAA, AA and water) in chamber after sampling.
Volume of the chamber (V): 120 L
R (gas constant)=62.363 L* torr*K⁻¹*mol⁻¹
Total mole of gases (air, H₂O₂, PAA, AA and water) collected n_{col}=n₁−n₂
Cold trap volume=10 mL DI
H₂O₂ collected mole=mole of H₂O₂ collected by cold trap in 10 mL DI
PAA collected mole (n_{PAA} collected)=mole of PAA collected by cold trap in 10 mL DI
AA collected mol=mole of AA collected by cold trap in 10 mL DI
Molecular weight of PAA=76.05 g/mol
   The calculations were as follows:

$$n 1 = \frac{P 1 * V}{RT}$$

$$n 2 = \frac{P 2 * V}{RT}$$

$${n}_{col} = {n}_{1} - {n}_{2}$$

Note: For the n₁ and n₂ calculation, the volume used was the volume of the chamber 702 which is constant at 120 L.
PAA vapor (mg/L) initial:

$$PAA vapor initial ( \frac{mg}{L} ) = \frac{nPAA collected * n 1 * 76.05 * 1000}{ncol * 120}$$

Table 2 shows the data for a first set of runs at 100 torr.

TABLE 2
Operation at 100 torr

| | | | | Initial | Final | Total | | | | | PAA | H2O2 | AA |
|---|---|---|---|---|---|---|---|---|---|---|---|---|---|
| In- | Initial | Final | | total | total | mol | H₂O₂ | PAA | AA | PAA-IR | Vapor | Vapor | Vapor |
| jection | Pressure | Pressure | Temp. | mol | mol | collected | collected | collected | collected | Abs. | mg/L | mg/L | mg/L |
| vol. | P1, torr | P2, torr | (T) | (n1) | (n2) | (ncol.) | mol | mol | mol | (highest) | (initial) | (initial) | (initial) |
| 0.5 mL | 99.6 | 80 | 22.34 | 0.64892 | 0.52122 | 0.12770 | 0.00000 | 0.00006 | 0.00013 | 0.01370 | 0.19889 | 0.00478 | 0.33726 |
| 1.0 mL | 100.4 | 80.34 | 22.95 | 0.65413 | 0.52344 | 0.13070 | 0.00000 | 0.00010 | 0.00031 | 0.02540 | 0.32256 | 0.00444 | 0.76525 |
| 1.5 mL | 100.26 | 81.08 | 22.66 | 0.65322 | 0.52826 | 0.12496 | 0.00001 | 0.00016 | 0.00052 | 0.03820 | 0.53968 | 0.01007 | 1.37150 |
| 2.0 mL | 100.62 | 81.03 | 22.16 | 0.65557 | 0.52793 | 0.12763 | 0.00005 | 0.00025 | 0.00080 | 0.05940 | 0.82900 | 0.07048 | 2.04406 |
| 2.5 mL | 100.47 | 81.37 | 23.04 | 0.65459 | 0.53015 | 0.12444 | 0.00001 | 0.00027 | 0.00069 | 0.07330 | 0.91433 | 0.01401 | 1.80428 |
| 3.0 mL | 100.62 | 82.01 | 23.18 | 0.65557 | 0.53432 | 0.12125 | 0.00001 | 0.00034 | 0.00085 | 0.08640 | 1.15710 | 0.01837 | 2.29261 |
| 3.5 mL | 100.62 | 87.21 | 22.8 | 0.65557 | 0.56820 | 0.08737 | 0.00000 | 0.00023 | 0.00041 | 0.09740 | 1.11306 | 0.00836 | 1.52239 |
| 4.0 mL | 100.47 | 87.96 | 23.04 | 0.65459 | 0.57308 | 0.08151 | 0.00000 | 0.00026 | 0.00042 | 0.10590 | 1.34080 | 0.00735 | 1.70164 |

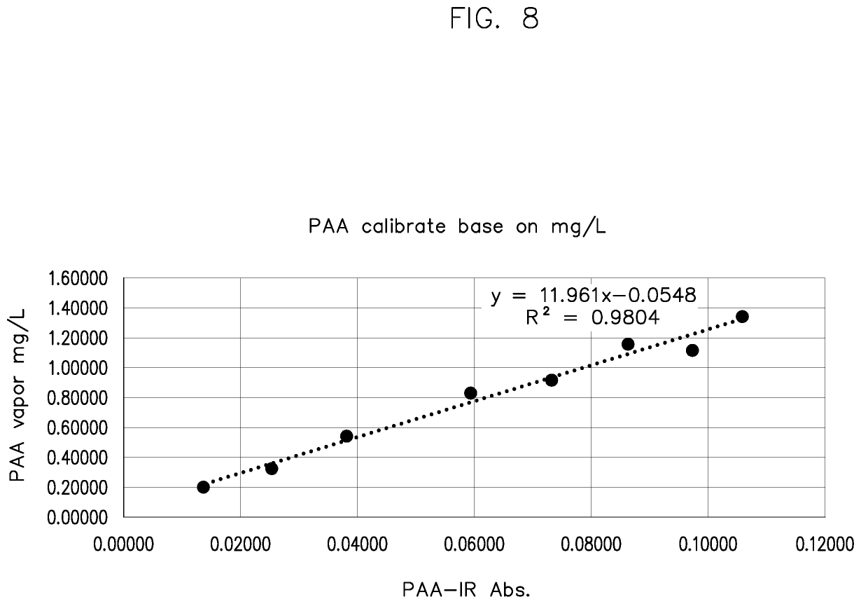

FIG. 8 shows a graph of the calculated PAA vapor (mg/L) and PAA absorption calibration curve with the injection volumes at operating pressure of 100 torr for the data in Table 2.

Table 3 shows the data for a second set of runs at 100 torr.

TABLE 3
Operation at 100 torr

| | Initial | Final | | Initial | Final | Total | | | | | PAA | | |
|---|---|---|---|---|---|---|---|---|---|---|---|---|---|
| | Pressure | Pressure | | (n1) | (n2) | mol | H2O2 | PAA | AA | PAA-IR | Vapor | H2O2 | AA |
| | (P1), | (P2), | Temp. | total | total | collect | collect | collect | collect | Abs. | mg/L | Vapor | Vapor |
| Run | torr | torr | (T) | mol | mol | (ncol) | mol | mol | mol | (highest) | (initial) | mg/L | mg/L |
| 0.5 mL | 100.62 | 80.8 | 22.8 | 0.65455 | 0.52561 | 0.12893 | 0.00009 | 0.00009 | 0.00026 | 0.02330 | 0.29858 | 0.12252 | 0.64790 |
| 1 mL | 100.4 | 80 | 22.75 | 0.65312 | 0.52041 | 0.13270 | 0.00016 | 0.00010 | 0.00032 | 0.02580 | 0.31208 | 0.22166 | 0.78443 |
| 1.5 mL | 100.33 | 80 | 23.03 | 0.65266 | 0.52041 | 0.13225 | 0.00031 | 0.00019 | 0.00066 | 0.04710 | 0.58562 | 0.42684 | 1.62181 |
| 2.0 mL | 100.69 | 80.57 | 23.9 | 0.65500 | 0.52412 | 0.13088 | 0.00018 | 0.00023 | 0.00071 | 0.05690 | 0.72406 | 0.25224 | 1.78902 |
| 2.5 mL | 100.19 | 80.1 | 23.3 | 0.65175 | 0.52106 | 0.13069 | 0.00032 | 0.00029 | 0.00095 | 0.07360 | 0.92836 | 0.44716 | 2.36121 |
| 3 mL | 100.47 | 81.2 | 23.3 | 0.65357 | 0.52822 | 0.12535 | 0.00054 | 0.00035 | 0.00110 | 0.08250 | 1.15755 | 0.79138 | 2.87761 |
| 3.5 mL | 100.76 | 88.33 | 23.2 | 0.65546 | 0.57460 | 0.08086 | 0.00018 | 0.00026 | 0.00067 | 0.08860 | 1.33489 | 0.40223 | 2.72997 |
| 4 mL | 100.47 | 88.21 | 23.1 | 0.65357 | 0.57382 | 0.07975 | 0.00016 | 0.00028 | 0.00069 | 0.09660 | 1.47256 | 0.37557 | 2.84564 |

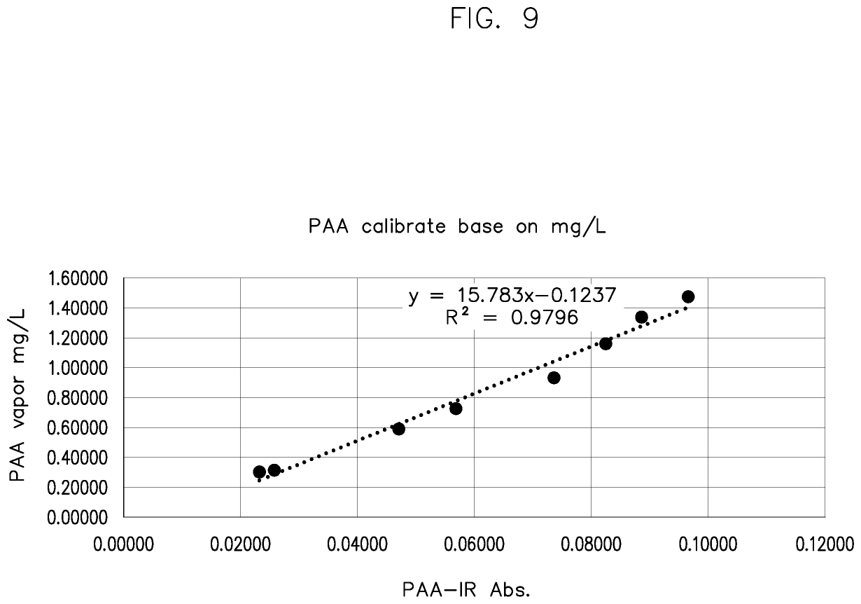

FIG. 9 shows a graph of the calculated PAA vapor (mg/L) and PAA absorption calibration curve with the injection volumes at operating pressure of 100 torr for the data in Table 3.

Table 4 shows the data for a first set of runs at 75 torr.

TABLE 4
Operation at 75 torr

| | | | | | | | | | | | PAA | | |
|---|---|---|---|---|---|---|---|---|---|---|---|---|---|
| | Initial | Final | | Initial | Final | Total | H2O2 | PAA | AA | PAA-IR | Vapor | H2O2 | AA |
| | Pressure | Pressure | | total | total | mol | collected | collected | collected | Abs. | mg/L | Vapor | Vapor |
| Run | P1, torr | P1, torr | Temp. | mol | mol | collected | mol | mol | mol | (highest) | (initial) | mg/L | mg/L |
| 1 mL | 75.49 | 61.75 | 22.75 | 0.53209 | 0.43524 | 0.09685 | 0.00025 | 0.00014 | 0.00042 | 0.03720 | 0.50242 | 0.39549 | 1.14824 |
| 2.0 mL | 75.7 | 62.01 | 23.9 | 0.53357 | 0.43707 | 0.09649 | 0.00023 | 0.00027 | 0.00072 | 0.06640 | 0.94882 | 0.36469 | 1.99867 |
| 3 mL | 75.81 | 62.32 | 23.3 | 0.53434 | 0.43926 | 0.09508 | 0.00018 | 0.00038 | 0.00088 | 0.09190 | 1.35501 | 0.29016 | 2.48425 |
| 4 mL | 92.88 | 78.88 | 23.1 | 0.65466 | 0.55598 | 0.09868 | 0.00013 | 0.00036 | 0.00070 | 0.10980 | 1.49787 | 0.24090 | 2.31235 |

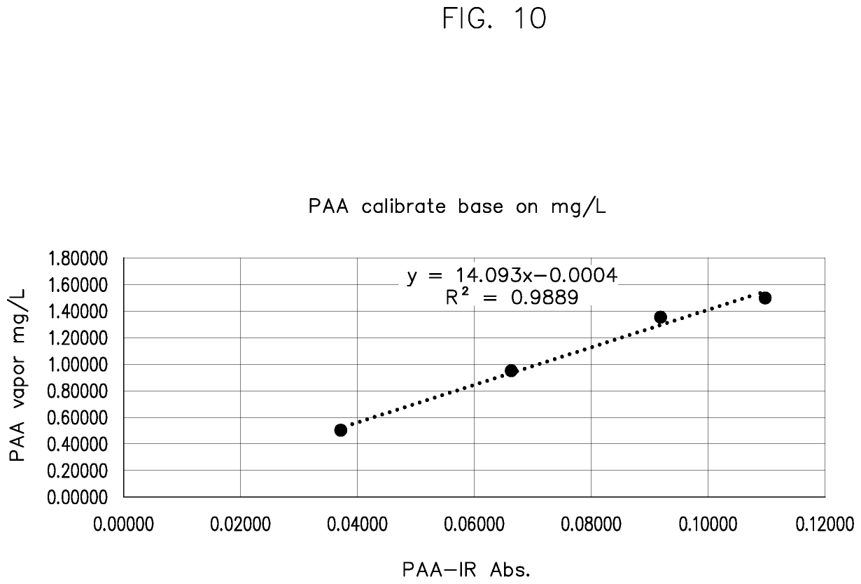

FIG. 10 shows a graph of the calculated PAA vapor (mg/L) and PAA absorption calibration curve with the injection volumes at operating pressure of 75 torr for the data in Table 4.

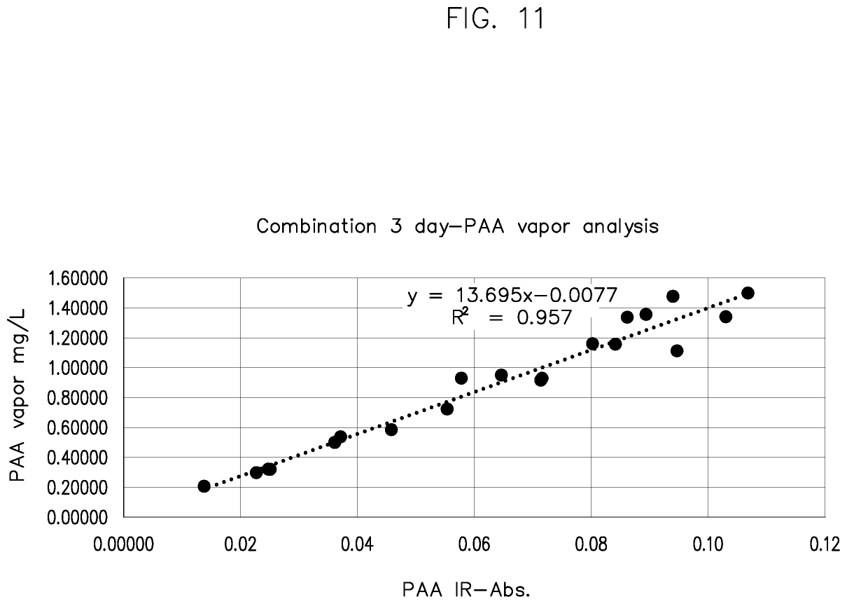

FIG. 11 shows a graph of the calculated PAA vapor (mg/L) and PAA absorption calibration curve with the injection volumes for the accumulated data from Tables 2, 3 and 4.

### Example 4

The purpose of this example was to calculate the PAA vapor (mg/L) concentration based on the PAA-IR calibration curve created in Example 3.

Material
Agilent pump, Model: G1310B 1260 IsoPump, SN: DEAB903915
Ivek nozzle (long nozzle, Sonicair 5 mm, Ext Tip, Encap 0.5 mm PN 14658⁻¹5)
Magtech % RH and Temperature sensor
Chemistry: an aqueous solution comprising approximately 5% PAA, 23% H₂0₂, 6% AA and the remainder water.
Balance, OHAUS Adventurer, SN: C5954
Vaporization chamber test bed with 120 L chamber
Timer
FTIR Detection Experiment:
   FTIR system, Thermo, Nicolet iS5, SN: ASB1817658
   10-meter gas cell
   PTFE tubing
FTIR program configuration:
   Number of Scans: 4
   Resolution: 4
   Absorbance mode
   Sample Compartment: Main
   Detector: DGTS KBr
   Beam Splitter: KBr
   Source: IR
   Accessory: iD Base Adaptet Plate for Nicolet iS5
   Window: ZnSe
   Range: 1600-800 cm⁻¹
   Gain: 8
   Optical Velocity: 0.4747
   Aperture: 100

Procedure

Before each run, the RH and temperature sensors were placed into the chamber. The test cycle was started by pumping the chamber down to 10 torr.

Once the chamber pressure reached 10 torr, the injection was started. The injection volumes that were tested are shown in Table 5 with the liquid flow rate and air flow rate used for the injection process.

TABLE 5

| Injection | Chemistry Liquid | Injection Air flow |
|---|---|---|
| Vol (mL) | flowrate (mL/min) | rate (L/min) |
| 2.5 | 2 | 3.5 |
| 2.5 | 2 | 3.5 |
| 2.5 | 2 | 3.5 |
| 2.5 | 2 | 3.5 |

Once the desired volume of chemistry was injected (based on the mass reading from the scale using the density of 1.18 g/mL), the liquid injection and the injection air were stopped.

Immediately after injections, the vacuum pump was turned on and FTIR chemistry sampling and cold trap sampling were collected for 5 min after the injection process was completed. The pressure of the chamber was recorded with each sampling time before and after the sampling process.

After the sampling process was completed, the venting process was begun. The chamber was vented with 4 venting cycle to remove the chemistry from the chamber.

IR analysis: For all IR absorbance data, the absorbance value obtained was defined as the highest absorbance observed at 860 cm⁻¹ during the vapor sample process.

Cold trap sample analysis: after leaving the IR chamber, the gas was collected in 10 mL of water via a cold trap. An HPLC method for organic acids was used to calculate PAA vapor concentration.

The PAA-IR Abs. and calibration curve y=13.695x-0.0077 (from FIG. 11 of Example 3) was then used to calculate the PAA vapor concentration. (x=PAA IR-Abs. and y=PAA vapor (mg/L)

Table 6 shows the calculation of PAA vapor with the measured PAA-IR absorption and cold trap method. The errors were less than 10% for 2.5 mL injection. The concentration of PAA vapor is about 1 mg/L.

TABLE 6

| | | | | | | | | | PAA | | |
|---|---|---|---|---|---|---|---|---|---|---|---|
| | | | | | | | | | Vapor | PAA | |
| | Initial | Final | | Initial | Final | Total | PAA | PAA-IR | mg/L | vapor | |
| Injection | Pressure, | Pressure, | | total | total | mol | collected | Abs. | (cold | mg/L | % |
| volume | torr | torr | Temp. | mol | mol | collected | mol | (highest) | trap) | (IR) | Error |
| 2.5 mL⁻¹ | 63.61 | 53.42 | 23 | 0.4135 | 0.3473 | 0.0662 | 0.0002 | 0.0714 | 0.9824 | 0.9705 | −1.2101 |
| 2.5 mL-2 | 53.37 | 46.32 | 22.75 | 0.3469 | 0.3011 | 0.0458 | 0.0002 | 0.0642 | 0.9169 | 0.8717 | −4.9320 |
| 2.5 mL-3 | 57.32 | 48.33 | 23 | 0.3726 | 0.3142 | 0.0584 | 0.0003 | 0.0741 | 1.0943 | 1.0071 | −7.9655 |
| 2.5 mL-4 | 63.7 | 52.31 | 23.9 | 0.4141 | 0.3401 | 0.0740 | 0.0003 | 0.0732 | 1.0701 | 0.9949 | −7.0270 |

The results above show that that it is possible to use the PAA-IR spectrum at wavelength 860 cm⁻¹ to detect PAA vapor concentration in a sterilizer chamber.

The error of the calculation was less than 10%. To improve the standard curve, a vapor filter could be used to avoid small liquid particles getting into sample apparatus.

In light of the detailed description of the invention and the examples presented above, it can be appreciated that the several objects of the invention are achieved.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

We claim:

1. A peracetic acid vapor and hydrogen peroxide vapor detection system, the system comprising
a. a source of peracetic acid vapor, hydrogen peroxide vapor, water vapor and acetic acid vapor:
b. a light source configured to supply light with at least a component in the mid-infrared range; and
c. a detector configured to individually detect mid-infrared range light in (a) a first mid-infrared spectrum absorbed by the peracetic acid vapor and not absorbed by the hydrogen peroxide vapor, the acetic acid vapor or the water vapor, and (b) a second mid-infrared spectrum absorbed by the peracetic acid vapor and the hydrogen peroxide vapor; wherein the first mid-infrared spectrum is from about 830 cm⁻¹ to about 880 cm⁻¹, and wherein the second mid-infrared spectrum is from about 1220 cm⁻¹ to about 1260 cm⁻¹.

2. The system of claim 1, wherein the detector further individually detects a third mid-infrared spectrum absorbed by the acetic acid vapor.

3. The system of claim 2, wherein the third mid-infrared spectrum is from about 1140 cm⁻¹ to about 1200 cm⁻¹.

4. The system of claim 1, further comprising a light source which supplies light with at least a component in the near-infrared range.

5. The system of claim 4, further comprising a detector which individually detects near-infrared range light in a near-infrared spectrum absorbed by the peracetic acid vapor, the hydrogen peroxide vapor and the acetic acid vapor.

6. The system of claim 5, wherein the near-infrared spectrum is from about 1390 nm to about 1430 nm.

7. The system of claim 1, further comprising; a processor configured to determine at least a concentration of the peracetic acid vapor from the detected light in the first mid-infrared spectrum.

8. The system of claim 7, wherein the processor is configured to determine at least a concentration of the hydrogen peroxide vapor from the detected light in the second mid-infrared range spectrum.

9. The system of claim 7, wherein the processor is configured to determine at least a concentration of the hydrogen peroxide vapor from the detected light in the near-infrared spectrum.

10. A peracetic acid and hydrogen peroxide treatment system comprising:
a. a treatment chamber;
b. a vaporizer configured for generating a mixture of peracetic acid vapor, hydrogen peroxide vapor, water vapor and acetic acid vapor and supplying the vapor mixture to the treatment chamber;
c. a light source configured to supply light to the treatment chamber with at least a component in the mid-infrared range;
d. a detector configured to individually detect mid-infrared range light in a first spectrum absorbed by peracetic acid vapor and not any of the hydrogen peroxide vapor, water vapor and acetic acid vapor, and a second spectrum absorbed by the peracetic acid vapor and the hydrogen peroxide vapor; wherein the first mid-infrared spectrum is from about 830 cm⁻¹ to about 880 cm⁻¹, and wherein the second mid-infrared spectrum is from about 1220 cm-1 to about 1260 cm⁻¹ and,
e. a processor configured to determine the concentration of the peracetic acid vapor in the treatment chamber.

11. The system of any of claim 10, wherein the detector further individually detects a third mid-infrared spectrum absorbed by the acetic acid vapor.

12. The system of claim 11, wherein the third mid-infrared spectrum is from about 1140 cm⁻¹ to about 1200 cm⁻¹.

13. The system of any of claim 10, further comprising a light source which supplies light with at least a component in the near-infrared range.

14. The system of claim 13, wherein the near-infrared spectrum is from about 1390 nm to about 1430 nm.

15. The system of any of claim 10, wherein the system is configured to treat a medical device.

16. A method for detecting the presence of peracetic acid and hydrogen peroxide in a vapor mixture, comprising the steps of
a. providing a vaporized mixture comprising peracetic acid, hydrogen peroxide, acetic acid, and water into a chamber;
b. projecting light in a mid-infrared range through a portion of the vaporized mixture that has passed through at least a portion of the chamber;
c. detecting mid-infrared light in a first spectrum absorbed by the peracetic acid vapor and not any of the hydrogen peroxide vapor, the acetic acid vapor and the water vapor, and a second narrow spectrum absorbed by a peracetic acid vapor and hydrogen peroxide vapor; and
d. detecting mid-infrared light in a second spectrum absorbed by the peracetic acid vapor and the hydrogen peroxide vapor; wherein the first mid-infrared spectrum is from about 830 cm⁻¹ to about 880 cm⁻¹, and wherein the second mid-infrared spectrum is from about 1220 cm-1 to about 1260 cm⁻¹ and.

17. The method of claim 16, further comprising determining at least a concentration of the peracetic acid vapor from the light detected in first spectrum.

18. The method of claim 16, further comprising determining a concentration of the hydrogen peroxide from the light detected in the second narrow spectrum.

19. The method of claim 16, further comprising converting the determined spectrums into one of absorbance and transmittance of mid infrared light through the vapor mixture.

20. The method of claim 19, further comprising converting the determined one of the absorbance and transmittance into the concentration of the peracetic acid vapor and a concentration of the hydrogen peroxide vapor.
